(19) United States
(12) Patent Application Publication
(10) Pub. No.: US 2006/0015184 A1
(43) Pub. Date: Jan. 19, 2006

(54) Stacking implants for spinal fusion

(76) Inventors: John Winterbottom, Jackson, NJ (US); Ryan Belaney, Old Bridge, NJ (US); David Knaack, Summit, NJ (US); Todd Boyce, Matawan, NJ (US); Lawrence Shimp, Morganville, NJ (US); Samuel Lee, Red Bank, NJ (US); David Kaes, Toms River, NJ (US); Marc Henry Burel, Towaco, NJ (US)

Correspondence Address: CHOATE, HALL & STEWART LLP, TWO INTERNATIONAL PLACE, BOSTON, MA, 02110, US

(21) Appl. No.: 11/047,982

(22) Filed: Jan. 31, 2005

Related U.S. Application Data
(60) Provisional application No. 60/540,375, filed on Jan. 30, 2004.

(51) Int. Cl. A61F 2/30

(52) U.S. Cl. 623/18.11; 424/426

(57) ABSTRACT

An implant system for fusing vertebrae includes a variety of shapes that may be stacked to accommodate different intervertebral spacings and curvatures. The implants comprise polymer-bone composites that have osteogenic properties. By selection of an appropriate set of shapes, the surgeon can tailor the overall shape of the implant before or during surgery, in order to best match the shape of the intervertebral cavity for a particular patient.

This application claims priority from U.S. Provisional Application No. 60/540,375, filed Jan. 30, 2004, the entire contents of which are incorporated herein by reference.

### FIELD OF THE INVENTION

The present invention relates to an implant system for fusing vertebrae, and in particular to a set of units that may be stacked to accommodate different intervertebral spacings and curvatures.

### BACKGROUND OF THE INVENTION

Spinal fusion is a well-known treatment for severe conditions of the intervertebral disc, such as chronic herniation or degenerative disc disease. Adjacent vertebrae may be fixed to one another while bone growth occurs by a variety of removable or permanent mechanical devices, such as pedicle screws (which fix the relationship of the pedicles of the adjacent vertebrae). Alternatively, a variety of permanent implants may be placed between the vertebrae, with or without the use of external anchoring devices. Examples of such implants may be found, for example, in U.S. Pat. No. 6,206,957 to Driessens et al., U.S. Pat. No. 6,241,771 to Gresser et al., U.S. Pat. No. 6,443,987 to Bryan, U.S. Pat. No. 6,447,544 to Michelson, and U.S. Pat. No. 6,454,807 to Jackson, the contents of all of which are incorporated here by reference.

It may be difficult or impossible to accurately measure the size and shape of the intervertebral cavity prior to surgery, so it is generally desirable for implants to have some degree of adjustability. A need still exists for an implant system that is easy for a surgeon to use, and that can be readily adjusted to accommodate individual physiological differences.

### SUMMARY OF THE INVENTION

In one aspect, the present invention comprises a system for inducing fusion of vertebrae. The system includes a plurality of stacking inserts for placement in an intervertebral space. Each insert comprises a composite with osteogenic properties, consisting essentially of bone fragments embedded in a biocompatible polymer. A subset of the plurality of inserts in the system may be selected to fit the dimensions of the intervertebral space. The biocompatible polymer may be biodegradable and/or electroactive, for example, collagen-GAG, collagen, oxidized cellulose, fibrin, elastin, starches, polylactic acid, polyglycolic acid, polylactic-co-glycolic acid, polylactide, polyglycolide, poly(lactide-co-glycolide), polydioxanone, polycarbonates, polyhydroxybutyrate, polyhydroxyvalyrate, poly(propylene glycol-co-fumaric acid), polyhydroxyalkanoates, polyphosphazenes, poly(alkylcyanoacrylates), degradable hydrogels, poloxamers, polyarylates, amino-acid derived polymers, amino-acid-based polymers, amino-acid-based polymers, tyrosine-based polymers, tyrosine-based polycarbonates and polyarylates, pharmaceutical tablet binders, polyvinylpyrrolidone, cellulose, ethyl cellulose, micro-crystalline cellulose and blends thereof, starch ethylenevinyl alcohols, poly(anhydrides), poly(hydroxy acids), poly(ortho esters), poly(propylfumerates), poly(caprolactones), polyamides, polyamino acids, polyacetals biodegradable polycyanoacrylates, biodegradable polyurethanes, natural and modified polysaccharides, recombinant versions of biological polymers, silk-elastin, polypyrrole, polyanilines, polythiophene, polystyrene, polyesters, non-biodegradable polyurethanes, polyureas, polyamides, poly(tetrafluoroethylene), poly(ethylene vinyl acetate), polypropylene, polyacrylate, polymethacrylate, poly(methyl methacrylate), polyethylene, poly(ethylene oxide), amino acid-derived polycarbonates, amino acid-derived polyarylates, polyarylates derived from certain dicarboxylic acids and amino acid-derived diphenols, anionic polymers derived from L-tyrosine, polyarylate random block copolymers, polycarbonates, poly(α-hydroycarboxylic acids), poly(caprolactones), poly(hydroxybutyrates), polyanhydrides, poly(ortho esters), polyesters, bisphenol-A based poly(phosphoesters), copolymers of polyalkylene glycol and polyester, or derivatives and combinations of any of the above. The bone particles may be nondemineralized, partially demineralized, or fully demineralized, and may comprise cortical bone, cancellous bone, cortico-cancellous bone, or mixtures thereof. The bone particles may be obtained from autogeneous bone, allogenic bone, xenogenic bone, or mixtures thereof, and may represent 50%-90%, 60%-80%, or 70%-75% of the composite by weight. At least some of the inserts may have parallel top and bottom surfaces, while others may have a wedge-shaped cross-section or may be in the form of a partial or complete spherical cap. The inserts may include connecting structures to inhibit relative movement between them (e.g., ridges, bumps, cylinders, pyramids, blocks, valleys, dimples, holes, grids, mortises, tenons, tongues, grooves, or dovetails), or securing structures to inhibit movement relative to adjacent vertebrae (e.g., ridges, bumps, cylinders, pyramids, blocks, valleys, dimples, holes, or grids). The system may also comprise one or more fasteners for connecting inserts to one another (e.g., screws, rivets, biscuits, rabbets, dowels, or extensible structures that lock around a set of inserts), in which case at least a portion of the inserts may comprise predrilled holes, slots, or notches sized to accommodate the fastener. The system may also comprise a pedicle screw that prevents relative motion of vertebrae forming the intervertebral space.

In another aspect, the present invention comprises a method of fusing vertebrae. The method includes inserting into an intervertebral space defined by the adjacent vertebrae a plurality of inserts that together match the size and shape of the intervertebral cavity. The inserts comprise a composite with osteogenic properties, consisting essentially of bone fragments embedded in a biocompatible polymer. The biocompatible polymer may be biodegradable and/or electroactive, for example, collagen-GAG, collagen, oxidized cellulose, fibrin, elastin, starches, polylactic acid, polyglycolic acid, polylactic-co-glycolic acid, polylactide, polyglycolide, poly(lactide-co-glycolide), polydioxanone, polycarbonates, polyhydroxybutyrate, polyhydroxyvalyrate, poly(propylene glycol-co-fumaric acid), polyhydroxyalkanoates, polyphosphazenes, poly(alkylcyanoacrylates), degradable hydrogels, poloxamers, polyarylates, amino-acid derived polymers, amino-acid-based polymers, amino-acid-based polymers, tyrosine-based polymers, tyrosine-based polycarbonates and polyarylates, pharmaceutical tablet binders, polyvinylpyrrolidone, cellulose, ethyl cellulose, micro-crystalline cellulose and blends thereof, starch ethylenevinyl alcohols, poly(anhydrides), poly(hydroxy acids), poly(ortho esters), poly(propylfumerates), poly(caprolactones), polyamides, polyamino acids, polyacetals biodegradable polycyanoacrylates, biodegradable polyurethanes, natural and modified polysaccharides, recombinant versions of biological polymers, silk-elastin, polypyrrole, polyanilines, polythiophene, polystyrene, polyesters, non-biodegradable polyurethanes, polyureas, polyamides, poly(tetrafluoroethylene), poly(ethylene vinyl acetate), polypropylene, polyacrylate, polymethacrylate, poly(methyl methacrylate), polyethylene, poly(ethylene oxide), amino acid-derived polycarbonates, amino acid-derived polyarylates, polyarylates derived from certain dicarboxylic acids and amino acid-derived diphenols, anionic polymers derived from L-tyrosine, polyarylate random block copolymers, polycarbonates, poly(α-hydroycarboxylic acids), poly(caprolactones), poly(hydroxybutyrates), polyanhydrides, poly(ortho esters), polyesters, bisphenol-A based poly(phosphoesters), copolymers of polyalkylene glycol and polyester, or derivatives and combinations of any of the above. The bone particles may be nondemineralized, partially demineralized, or fully demineralized, and may comprise cortical bone, cancellous bone, cortico-cancellous bone, or mixtures thereof. The bone particles may be obtained from autogeneous bone, allogenic bone, xenogenic bone, or mixtures thereof, and may represent 50%-90%, 60%-80%, or 70%-75% of the composite by weight. At least some of the inserts may have parallel top and bottom surfaces, while others may have a wedge-shaped cross-section or may be in the form of a partial or complete spherical cap. The inserts may include connecting structures to inhibit relative movement between them (e.g., ridges, bumps, cylinders, pyramids, blocks, valleys, dimples, holes, grids, mortises, tenons, tongues, grooves, or dovetails), or securing structures to inhibit movement relative to adjacent vertebrae (e.g., ridges, bumps, cylinders, pyramids, blocks, valleys, dimples, holes, or grids). The method may also include placing one or more fasteners for connecting inserts to one another (e.g., screws, rivets, biscuits, rabbets, dowels, or extensible structures that lock around a set of inserts), in which case at least a portion of the inserts may comprise predrilled holes, slots, or notches sized to accommodate the fastener. The method may also comprise placing a pedicle screw that prevents relative motion of the adjacent vertebrae.

### DEFINITIONS

The term “biomolecules”, as used herein, refers to classes of molecules (e.g., proteins, amino acids, peptides, polynucleotides, nucleotides, carbohydrates, sugars, lipids, nucleoproteins, glycoproteins, lipoproteins, steroids, lipids, etc.) that are commonly found in cells and tissues, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active subunits. In some embodiments, the biomolecule is a growth factor, cytokine, extracellular matrix molecule or a fragment or derivative thereof, for example, a cell attachment sequence such as RGD.

The term “biocompatible”, as used herein, is intended to describe materials that, upon administration in vivo, do not induce undesirable long term effects.

As used herein, “biodegradable”, “bioerodable”, or “resorbable” materials are materials that degrade under physiological conditions to form a product that can be metabolized or excreted without damage to organs. Biodegradable materials may be hydrolytically degradable, may require enzymatic action to fully degrade, or both. Other degradation mechanisms, e.g., thermal degradation due to body heat, are also envisioned. Biodegradable materials also include materials that are broken down within cells. Degradation may occur by hydrolysis, enzymatic degradation, phagocytosis, or other methods. “Polynucleotide”, “nucleic acid”, or “oligonucleotide”: The terms “polynucleotide,” “nucleic acid,” or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluroribose, ribose, 2′-deoxyriboses, arabinose, and hexose), or modified phosphate groups (e.g., phosphorothioates and 5′-N-phosphoramidite linkages). The polymer may also be a short strand of nucleic acids such as siRNA.

“Polypeptide”, “peptide”, or “protein”: As used herein, a “polypeptide”, “peptide”, or “protein” includes a string of at least two amino acids linked together by peptide bonds. The terms “polypeptide, “peptide”, and “protein”, may be used interchangeably. Peptide may refer to an individual peptide or a collection of peptides. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide”, as used herein, refer to any polymer or oligomer of carbohydrate residues. The polymer or oligomer may consist of anywhere from two to hundreds to thousands of sugar units or more. “Oligosaccharide” generally refers to a relatively low molecular weight polymer, while “starch” typically refers to a higher molecular weight polymer. Polysaccharides may be purified from natural sources such as plants or may be synthesized de novo in the laboratory. Polysaccharides isolated from natural sources may be modified chemically to change their chemical or physical properties (e.g., phosphorylated, cross-linked). Carbohydrate polymers or oligomers may include natural sugars (e.g., glucose, fructose, galactose, mannose, arabinose, ribose, and xylose) and/or modified sugars (e.g., 2′-fluororibose, 2′-deoxyribose, and hexose). Polysaccharides may also be either straight or branch-chained. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, and fructose. Glycosaminoglycans are also considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

“Small molecule”: As used herein, the term “small molecule” is used to refer to molecules, whether naturally-occurring or artificially created (e.g., via chemical synthesis), that have a relatively low molecular weight. Typically, small molecules have a molecular weight of less than about 5000 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body.

As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants (e.g., cyclosporine), anti-viral agents, enzyme inhibitors, neurotoxins, opioids, hypnotics, anti-histamines, lubricants, tranquilizers, anti-convulsants, muscle relaxants and anti-Parkinson agents, anti-spasmodics and muscle contractants including channel blockers, miotics and anti-cholinergics, anti-glaucoma compounds, anti-parasite, anti-protozoal, and/or anti-fungal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, targeting agents, neurotransmitters, proteins, cell response modifiers, and vaccines. In a certain preferred embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formular-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the FDA under 21 C.F.R. §§330.5, 331 through 361, and 440 through 460 and drugs for veterinary use listed by the FDA under 21 C.F.R. §§500 through 589, all of which is incorporated herein by reference, are also considered acceptable for use in accordance with the present invention.

The term “shaped” as applied to the osteoimplant herein refers to a determined or regular form or configuration, in contrast to an indeterminate or vague form or configuration (as in the case of a lump or other solid mass of no special form) and is characteristic of such materials as sheets, plates, blocks, cubes, spheres, disks, cones, pins, screws, tubes, teeth, bones, portion of bone, wedges, cylinders, threaded cylinders, and the like.

The phrase “wet compressive strength” as utilized herein refers to the compressive strength of the osteoimplant after the osteoimplant has been immersed in physiological saline (water containing 0.9 g NaCl/100 ml water) for a minimum of 12 hours and a maximum of 24 hours. Compressive strength is a well known measurement of mechanical strength.

The term “osteogenic” as applied to the osteoimplant of this invention shall be understood as referring to the ability of the osteoimplant to enhance or accelerate the ingrowth of new bone tissue by one or more mechanisms such as osteogenesis, osteoconduction and/or osteoinduction.

As utilized herein, the phrase “superficially demineralized” as applied to the bone particles refers to bone particles possessing at least about 90 weight percent of their original inorganic mineral content. The phrase “partially demineralized” as applied to the bone particles refers to bone particles possessing from about 8 to about 90 weight percent of their original inorganic mineral content, and the phrase “fully demineralized” as applied to the bone particles refers to bone particles possessing less than about 8, preferably less than about 1, weight percent of their original inorganic mineral content. The unmodified term “demineralized” as applied to the bone particles is intended to cover any one or combination of the foregoing types of demineralized bone particles.

Unless otherwise specified, all material proportions used herein are in weight percent.

### BRIEF DESCRIPTION OF THE DRAWING

The invention is described with reference to the several figures of the drawing, in which,

FIG. 1 is a schematic illustrating stacking units according to one embodiment of the invention being inserted into an intervertebral cavity.

FIG. 2 is a schematic of the arrangement of units in stacks according to various embodiments of the invention.

FIG. 3 is a schematic diagram illustrating the placement of a cap-shaped unit on top of horizontally stacked units.

FIG. 4 is a schematic diagram illustrating a variety of exemplary structures that may be incorporated into stacking units to inhibit relative motion.

FIG. 5 is a schematic diagram illustrating exemplary shapes for stacking units according to some embodiments of the invention.

FIG. 6 includes schematic diagrams of stacking units according to exemplary embodiments of the invention.

FIG. 7 is a schematic diagram of stacking units according to an exemplary embodiment of the invention.

FIG. 8 is a schematic diagram illustrating stacking units according to an embodiment of the invention.

FIG. 9 is a schematic diagram of stacking units according to an embodiment of the invention.

### DETAILED DESCRIPTION

In one embodiment, spinal implants according to the invention comprise a plurality of stacking units formed from an osteogenic composite material comprising bone or ceramic fragments embedded in a biocompatible polymer matrix. The details of materials for the stacking units are set forth infra. These materials have excellent osteogenic properties, and can eliminate the need to harvest bone from the patient for autografts.

The cranial and caudal surfaces of the body of a vertebra are generally concave (to accommodate the intervertebral discs), with a curvature that varies significantly from individual to individual and from vertebra to vertebra within the spine. In addition, different areas of the spine will have differing degrees of lordosis (backwards curvature) and kyphosis (forwards curvature). The present inventors have recognized that these physiological differences can be accommodated by a system of stacking disks, optionally including wedges and “caps” (solids formed by the intersection of a sphere or other curved body and a secant plane). By using stacking units as “shims” to correctly place the vertebrae, the surgeon can achieve a near-perfect fit without needing to construct a specially shaped implant in advance of surgery.

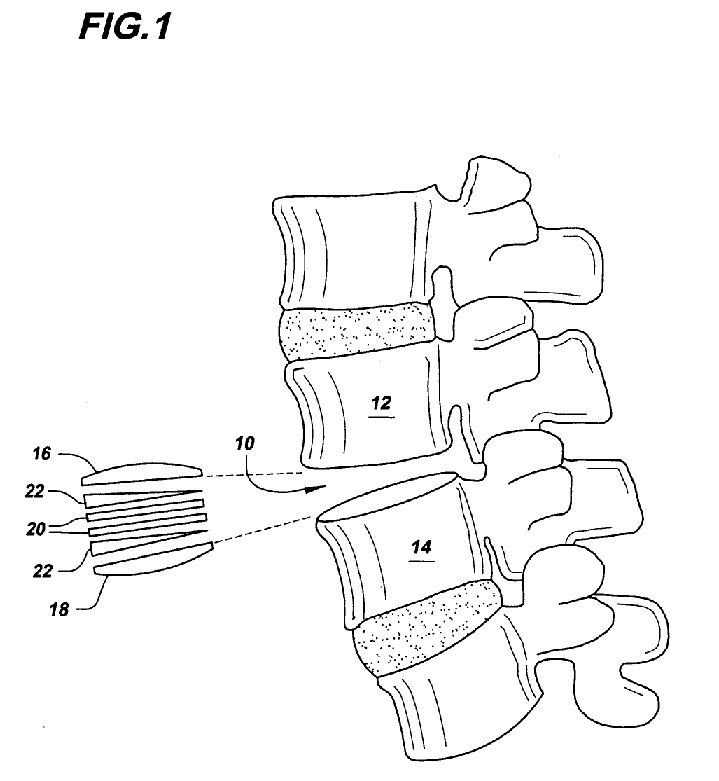

FIG. 1 shows a segment of the lumbar spine, with a set of stacking units according to one embodiment of the invention. As shown, an anterior approach is being used for surgery, but posterior and lateral approaches are also possible, and may be preferred in some situations. The stacking units may be placed in intervertebral space 10 in order to promote fusion of vertebrae 12 and 14. Two “cap” style pieces 16 and 18 may be selected to fit the caudal and cranial surfaces of vertebrae 12 and 14 respectively. Note that these caps need not have the same curvature or size. Caps may also be partial (e.g., a half or a quarter of a full cap) so that several may be placed on top giving a custom fit. Further, if there is minimal curvature of the surfaces of the vertebral bodies, flat pieces may be used instead of caps. Flat disks smaller than the full width of the intervertebral space may also be used to compensate for curvature of the vertebral surface. In an alternative embodiment, the stacking units may be used to replace an entire vertebra, filling the space between the remaining vertebrae on either side. Because the inventive implants may be used to replace an intervertebral body or a vertebral body, the term “intervertebral” is used herein to describe the space between two consecutive vertebrae. If a vertebra is missing, the two vertebrae may not be adjacent. For example, if L3 is removed, an intervertebral implant may be inserted between L2 and L4 using the teachings of the invention. In this example, the intervertebral space refers to the space between L2 and L4 where L3 had been.

Flat stacking units 20 may be inserted in order to achieve the correct intervertebral spacing. As illustrated in FIG. 1, two such units are shown; more or fewer may be used as appropriate for any particular fusion operation. One advantage of the implant system of the invention is that the exact number of stacking units is adjustable for a particular surgery. In addition, stacking units of different thicknesses may be provided for a surgeon to achieve this adjustability. It is not necessary to provide a large set of one-piece implants to a surgeon to cover all possible intervertebral anatomies. Rather, a kit containing a far more limited set of “building blocks” of various sizes and shapes will allow a surgeon to construct an implant of the proper size and shape for a particular patient.

Wedge-shaped stacking units 22 may also be inserted between the other implants of the system. These units are used to replicate the proper lordosis of the spine, to avoid placing any stress on the spinal column during or after surgery. Of course, kyphosis may also be created, if appropriate, by reversing the direction of the wedge units. Not all types of stacking unit shapes may be required for all surgeries. In portions of the spine where curvature is minimal, wedge-shaped units may be eliminated. As discussed above, caps may also be eliminated or replaced with partial caps or smaller flat disks.

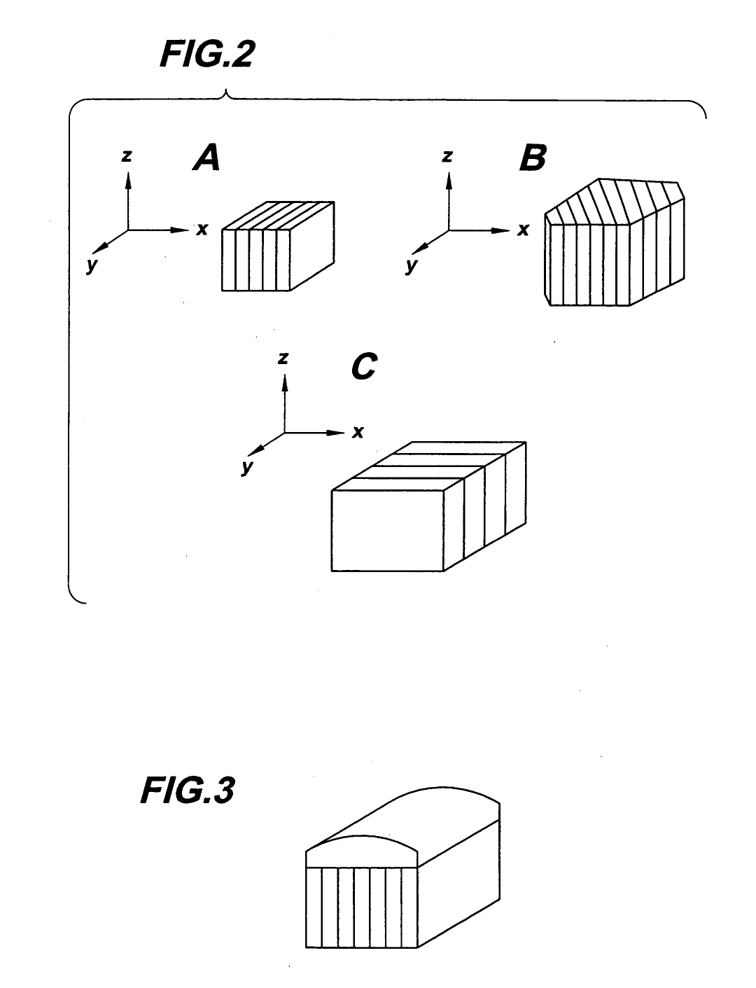

FIG. 1 gives one embodiment of both implant shape and stacking orientation. In other embodiments, the units may be stacked horizontally or diagonally. Horizontally stacked units may be stacked so an axis perpendicular to the stacked units is oriented roughly in the anterior-posterior direction or in a lateral direction, as shown in FIG. 2, regardless of the surgical approach. As shown in FIG. 2, the caudal-cranial axis is z, the anterior-posterior axis is y, and the lateral direction (towards the patient's right and left) is x. Horizontally or diagonally stacked units need not be symmetric. Because the caudal and cranial surfaces of the intervertebral space are not necessarily contoured in the same manner, the curvature of the upper and lower surfaces of horizontally or diagonally stacked units may benefit from not being the same. In an alternative embodiment, cap shaped and or/partial cap-shaped stacking units are inserted above and below horizontally or diagonally stacked stacking units (FIG. 3).

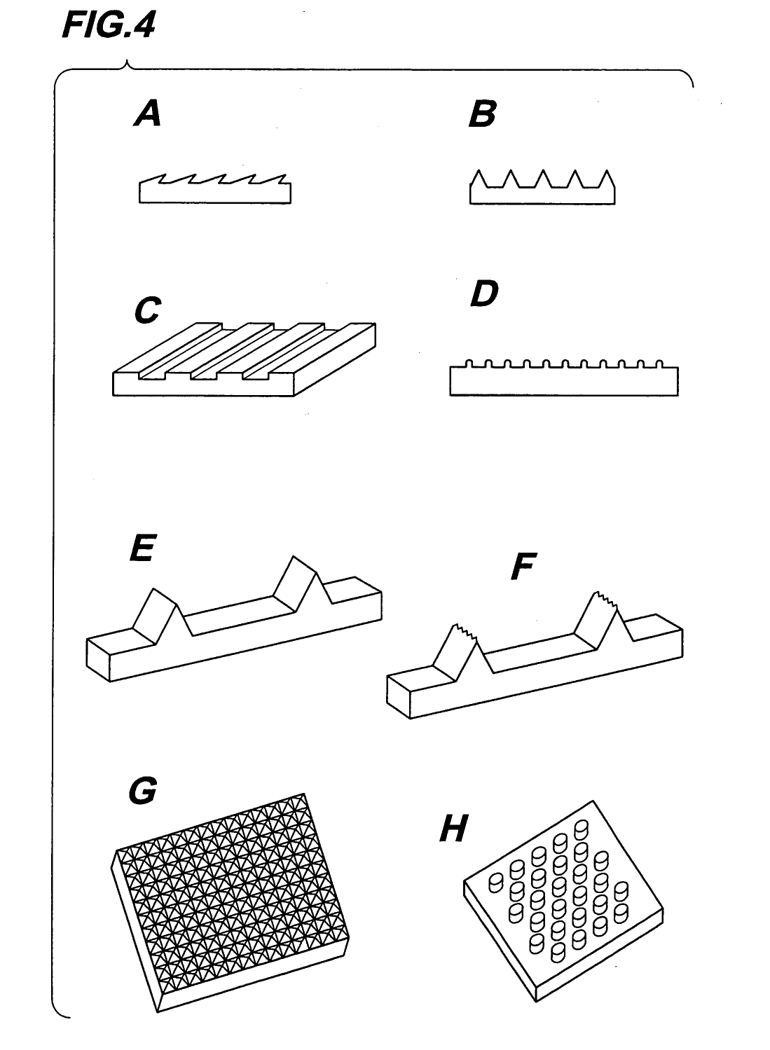

As shown in FIG. 1, the stacking disks, caps, and wedges have at least one flat side, so that they may be most easily inserted one-by-one into the intervertebral space. In other embodiments, individual stacking units may include mechanical structures to inhibit relative movement of the units in the intervertebral space, reducing the possibility of expulsion. These movement inhibitory structures may be in the form of three-dimensional, independent, discrete or continuous protrusions of any shape, with regular, irregular and/or random dispersion using a single shape or a combination of two or more shapes. For example, raised ridges, teeth, threads, wedges, bumps, cylinders, pyramids, and blocks or recessed structures such as valleys, dimples, holes and grids can be utilized. The raised portions themselves may be smooth or textured. For example, raised wedges may also be jagged. Of course, the angles and orientation of the texturing may also be varied. Once inserted into the intervertebral space, compression of the vertebrae on the unit's protrusions and/or recesses engages them with the opposing surface. Some exemplary textures are shown in FIG. 4.

Stacking units may also be connected by means of fastenerless mechanisms, interconnecting/complementary protrusions and recesses present on the individual units. The protrusions may be in the form of three-dimensional independent discrete or continuous projections of any shape with regular, irregular and/or random dispersion using a single shape or a combination of two or more shapes, for example, raised ridges, bumps, cylinders, pyramids, pegs, plugs, and blocks. The recesses of each interconnecting unit may be in the form of three-dimensional independent discrete or continuous cavities of any shape with regular, irregular and/or random dispersion using a single shape or a combination of two or more shapes. For example recessed structures such as valleys, troughs, dimples, pits, holes and grids can be utilized. In an alternative embodiment, the use of complementary protrusions and recesses may be combined with mechanisms that require rotation or other motion, such as threads and bayonet locks. Some examples of these are shown in FIG. 4.

The texturing of surfaces that abut one another need not be the same as the texturing of surfaces that abut the caudal and cranial surfaces of the intervertebral space. For example, an interlocking mechanism, such as complementary protrusions and grooves, may be used to hold the stacking units together, while a different surface texture, for example, bumps or rows of teeth, may be used to increase the friction of the stacking units against the surrounding tissue. Additional friction/interference fitting protrusions known to those skilled in the art may also be used to create an interlock between adjacent stacking units.

When stacking units are to be inserted one-by-one into the intervertebral space, they may nevertheless still include mechanical structures to permit the assembly of independent units into a single mass during or after insertion. For example, a tongue-in-groove geometry may be used to allow each stacking unit to slide along a fixed track into the intervertebral space, or the stacking units themselves may comprise a tongue-in-groove geometry so that they slide along the previously inserted member and interlock with it. Alternatively, the end cap units may include extensible structures allowing them to be “locked” around the plate and wedge units between them to hold all units together via interference fit once insertion is complete. Alternatively or in addition, stacking units may include protrusions that extend outside the intervertebral space. These protrusions may be bolted to brackets or plates after insertion. Conversely, brackets, plates, or braces such as those used in traditional spinal fusion techniques may be screwed or riveted to the stacking units to hold the implant units together. This allows the stacking units to behave as a unitary whole, engaging a large footprint on adjacent vertebrae and exhibiting the mechanical properties of a bulk implant while obviating the large incision that would be necessary to insert a full sized implant. Instead, the mechanical benefits of a large implant may be achieved along with the medical benefits of being able to insert individual implant components through a smaller incision.

When a recessed surface of a stackable interconnecting unit is contacted to a protruding surface of another stackable unit, the two surfaces may be engaged by simply setting one unit onto or next to the other and pressing the units together: by hand, by tapping them with a hammer, by using a general instrument (e.g., pliers), or by using a custom instrument specifically designed to engage the stackable interconnecting units. In some embodiments the interconnecting units may “snap” or “click” into each other, giving the surgeon positive tactile and/or auditory feedback of a successful connection. Additionally, the stacking units may also remain loosely associated through their complementary protrusions and recesses. In some embodiments the interconnecting units may be separated and reconnected with each other repeatedly, permitting the surgeon to continuously fit or adjust the units in a stack to obtain the desired effect.

In some embodiments, where a fastener is used to secure the stacked components of an implant, the stackable units may contain through bores that are offset from unit to unit. Before, during, or after stacking, pins or pegs may be inserted into these through bores, which hold the stacked implant together through friction created between the pins or pegs and the through bore side walls.

Adjacent stacking units may also be chemically connected. For example, chemical cross-linkers may be disposed on adjacent stacking units and reacted with one another after implantation. In some embodiments, the exposure to either physiological pH or temperatures may cause the cross-linkers to react with one another. In other embodiments, the stacking units may be exposed to an energy source to promote the formation of chemical links. For example, stacking units may be irradiated, for example, with microwave or ultraviolet radiation. Alternatively, enzymatic crosslinking agents may be employed. In some embodiments, metal ions may be used to form a bridge between adjacent stacking units. The use of metal ions to form bridges between adjacent ceramic particles is described below. Other chemical methods of connecting adjacent ceramic particles in a composite, such as those disclosed in U.S. Pat. Nos. 6,123,731 and 6,478,825, the entire contents of both of which are incorporated herein by reference, may be exploited to produce chemical linkages between stacking units. Adhesives may also be employed to connect stacking units. Exemplary adhesives include but are not limited to cyanoacrylates; epoxy-based compounds, dental resin sealants, dental resin cements, glass ionomer cements, polymethyl methacrylate, gelatin-resorcinol-formaldehyde glues, collagen-based glues, inorganic bonding agents such as zinc phosphate, magnesium phosphate or other phosphate-based cements, zinc carboxylate, etc., and protein-based binders such as fibrin glues and mussel-derived adhesive proteins.

In other embodiments of the invention, the disks may be assembled into a single unit before placement into the intervertebral space. Known fastenerless geometries such as bridle joints, cross-halving joints, tee halving joints, dovetail halving joints, half lap joints, lapped joints, finger joints, dovetail joints, mortise-and-tenon joints, or friction/interference fitting protrusions may be used to secure a set of disks into a single unit, or fasteners may be used to secure the disks into a single stacking unit. Some of these joints may be appropriate for linking stacking units inserted one-by-one instead of as an assembled unit. Alternatively, stacking units may be fabricated to receive fasteners that are used to connect stacking units after one-by-one insertion. Fasteners may include without limitation screws, rivets, biscuits, rabbets, and dowels, and the inserts may, but need not, include predrilled holes, slots, or notches for ease of fastener insertion. Of course, the interconnecting protrusions, adhesives, chemical links, and other fastening mechanisms described herein may also be used to assemble stacking units into a complete unit before implantation. While one-by-one insertion of stacking units enables insertion of the implant through a smaller incision, since a surgeon only needs to be able to fit a portion of the implant through the incision at a time, other patients may benefit from surgical techniques in which the surgeon has more expansive access to the intervertebral space.

In some embodiments of the invention, the stacked units may be slightly compressed by the vertebrae, so that no additional hardware is required to hold the vertebrae in a constant relationship while fusion occurs. In other embodiments, pedicle screws or other surgically placed holding devices known in the art may be used to prevent relative motion of the vertebrae until fusion occurs. In still other embodiments, an external device such as a back brace may be used to immobilize the vertebrae during fusion.

The stacking units themselves may be fabricated in a variety of shapes. The stacking units need not define symmetric shapes. For example, depending on the direction from which the stacking units are being loaded into the implant site, it may be desirable that the individual stacking units be curved on one side and flat on the other. Stacking units may be wedge-shaped in one or more of the caudal-cranial axis, posterior-anterior axis, or lateral axis. Alternatively or in addition, stacking units may be regularly shaped but include a taper at one side.

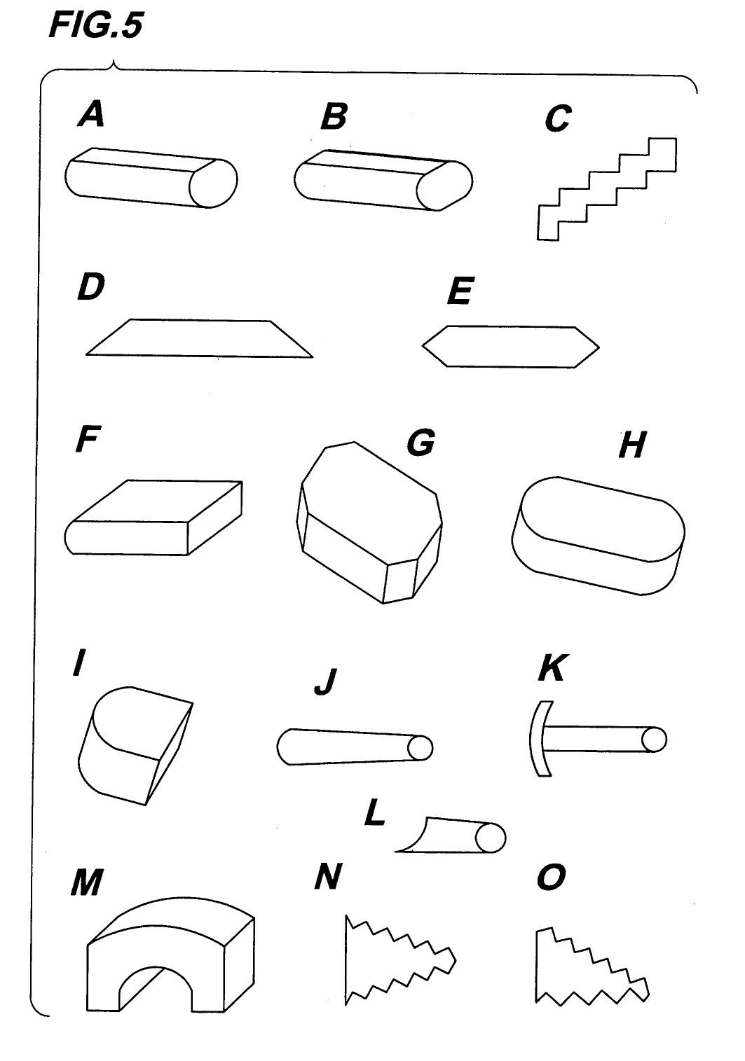

One common shape for prior art implants is an elongated polygon, rounded polygon, or oval shape having a bridge across the short axis of the implant unit. These implants are frequently fabricated from metals. After assembly, they are filled with a bone substitute material or other substance that can be degraded and replaced with endogenous tissue. One advantage of the present invention is that stacking units having these general shapes may be produced as solids. There is no need to leave a metal cage permanently disposed in the spinal column, where it may fatigue and crack. Rather, a biodegradable solid implant is employed that is able to bear weight almost immediately and that is entirely replaced by endogenous tissue. Some exemplary shapes for stacking units are shown in FIG. 5.

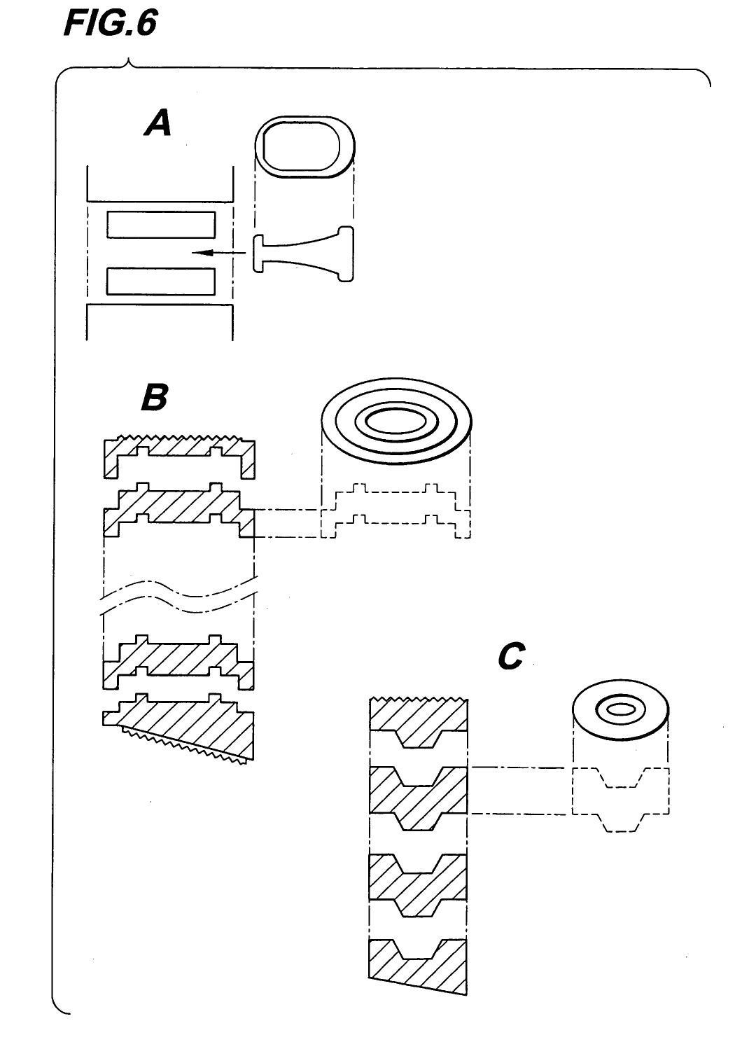

Because the stacking units may be fabricated as solid pieces, larger interconnecting protrustions may be used to connect adjacent stacking units than in prior art implants. In addition, these protrusions may be shaped so that they do not interlock until the unit is in place. Examples of these are given in FIGS. 6A, B, and C. The stacking units in these figures may be produced in different thicknesses to ease the assembly of implants in differently sized sites. Additional examples of interconnects that may be used to link adjacent stacking units include those described in U.S. Pat. Nos. 6,025,538 and 6,200,347, the entire contents of which are incorporated herein by reference. Additional configurations of stacking units include those disclosed in our co-pending application published as U.S. Patent Publication No. 20031055528, the contents of which are incorporated herein by reference.

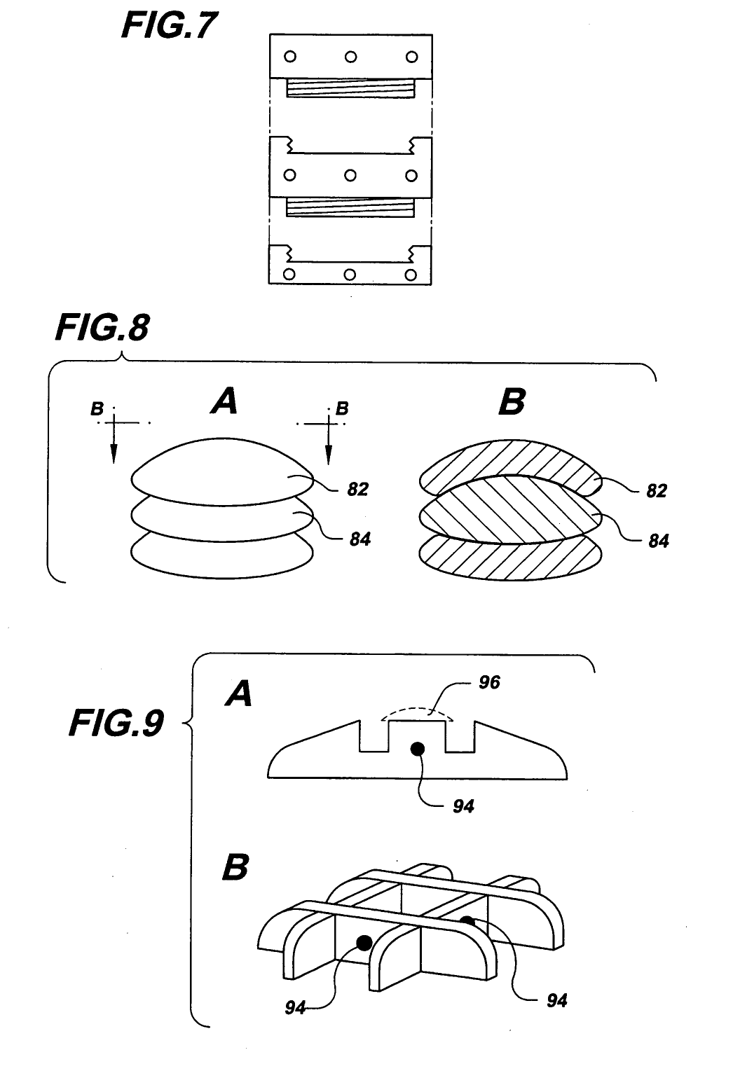

In another embodiment, stacking units may be fabricated with threaded surfaces. As shown in FIG. 7, the end pieces 70 and 72 have threaded surfaces. A central unit 74 includes the mating threads for the two implants. After the three pieces are in place, the central unit 74 is rotated with respect to the ends to engage the threads. Holes 76 may be included in one or more of the end pieces 70 and 72 and central unit 74 to facilitate rotation. In addition, FIG. 7 shows only three stacking units, but one skilled in the art will recognize that the assembled implant may include additional stacking units if desired.

FIG. 6A is one example of a self-distracting implant. Once a disk or vertebra has been removed, the remaining tissue in the spinal column tends to crowd the space from which the tissue has been removed. In some embodiments, implants according to the invention are self-distracting. In FIG. 6A, endcaps 60 and 62 are pressed up and down by the raised ridge 64 on central unit 66. It is not necessary for the surgeon to physically hold the endpieces apart in order to insert central unit 66. The wedges shown in FIG. 1 serve the same purpose. In another embodiment, a stacking unit for a self-distracting implant may have a wedged end or circumference and a central section having a uniform height. The wedge helps the surgeon initially insert the stacking unit into the available space and tap the implant unit into place. As it is pushed into position, the wedge helps push the material on either side apart to hold the surrounding tissue at the proper distance. Grooves in the mating surfaces of the wedge or partial wedge and the adjacent implant units, oriented perpendicular to the direction from which the wedge is pushed into the intervertebral space, help prevent the wedge from being ejected from the implant site by the compressive force of the surrounding material.

In another embodiment, a screw may be employed to adjust the height of a stack of units. For example, adjacent stacking units may be fabricated with complementary threaded or smooth grooves that mate to form a hole. A screw having a tapered end and a diameter larger than that of the hole may be used to push the stacking units apart. A set of screws may be used to minimize the amount of empty space between the stacking units and to distribute the compressive force over a larger area. Alternatively or in addition, a bone substitute material may be injected into the space on either side of the screws.

Stacking units may be designed to be inserted in any order, sequentially or non-sequentially. For example, the central units of an implant stack may be inserted first, followed by endcaps abutting the adjacent vertebral endplates, or vice versa. Both of these examples may be used in self-distracting implants. For example, FIG. 8 depicts an implant using roughly hemispherical shells 82 that conform to the endplates and a central, lens-shaped unit 84. Either the shells 82 or the central unit 84 may be inserted into the implant site first. Any of the interconnecting/complementary protrusions described above may be used to prevent relative motion of the components.

In some embodiments, it may be desirable to combine stacking units produced from composites with other materials. In one embodiment, stacking units are formed from both ceramic or bone-polymer composites and allograft bone. The allograft bone implants may be used in the central portions of the stack, while the composite units are used on either side of the allograft implant. The composite portions attract cells and are remodeled quickly, while the allograft implant contributes early mechanical strength and is of a size that it can be remodeled to endogenous bone before it fails through fatigue. In other embodiments, the composite stacking units described herein are used in combination with cage type implants such as those disclosed in U.S. Pat. Nos. 6,447,547; 6,443,987; 6,368,351; 6,371,986; 5,593,409; 5,865,848; 6,080,193; 6,251,140; 6,344,057; 6,159,211; 5,522,899; 6,447,544; 6,241,771; 6,409,765; 6,200,347; 6,025,538; U.S. Patent Publication No. 20020106393, PCT Publication No. WO01/70139, the contents of all of which are incorporated herein by reference.

In another embodiment, the stacking units may be formed with a hollow space to allow the injection of an osteogenic material, for example α-BSM (Etex Corp), Norian SRS (Norian Corp.), Grafton (Osteotech), Dynagraft (Citagenix), or the formable material disclosed in U.S. Patent Publication No. 20050008672. One example of this is shown in FIG. 9. Notched complementary units are fit together using a cross-halving joint. The ends of the units may be curved to allow the upper units to be slid over the lower units. The central “courtyard” defined by the units may be filled with a bone substitute material either during assembly or by injection through ports 94. The central portion 96 of the units may be curved to conform with the endplate or flat, if the endplate has been suitably prepared. Alternatively or in addition, stacks of these units may be inserted into the intervertebral space.

In another embodiment, otherwise solid stacking units may be produced with a small central hole, and a port may be provided to give access to the central column that results from stacking units vertically, either using specially molded stacking units or by simply drilling a hole. This central column is then filled with an injectable osteogenic material. The material overflows into the space between the assembled implant and the endplates, correcting any failure of the implant and the endplates to exactly conform with one another.

It may also be desirable to include stacking units of varying mechanical properties. For example, some stacking units may be prepared to be very hard and rigid, and these may be interspersed with more flexible units, for example, fabricated from composites with a lower proportion of ceramic or bone particles. Alternatively or in addition, polymer stacking units may be interspersed with composite stacking units, or thick layers of any of the adhesives discussed above may be interspersed between stacking units. Such implant stacks may provide a better approximation to the mechanical properties of a vertebral unit. Descriptions of other materials that may be interspersed between stacking units may be found in U.S. Pat. No. 5,899,939, the contents of which are incorporated herein by reference.

Materials

The bone particles employed in the preparation of the bone particle-containing composition can be obtained from cortical, cancellous and/or corticocancellous bone which may be of autogenous, allogenic and/or xenogeneic origin and may or may not contain cells and/or cellular components. In one embodiment, the bone particles are obtained from cortical bone of allogenic origin. Porcine and bovine bone are particularly advantageous types of xenogeneic bone tissue which can be used individually or in combination as sources for the bone particles.

Bone particles may be obtained by milling or shaving sequential surfaces of an entire bone or relatively large section of bone. A non-helical, four fluted end mill may be used to produce fibers having the same orientation as the milled block. Such a mill has straight grooves, or flutes, similar to a reamer, rather than helical flutes resembling a drill bit. During the milling process, the bone may be oriented such that the natural growth pattern (along the long axis) of the piece being milled is along the long axis of the end mill of the milling machine. Multiple passes of the non-helical end mill over the bone results in bone fibers having a long axis parallel to that of the original bone. (FIG. 1). As described herein, bone fibers are particles having at least one aspect ratio of 2:1 or greater. In some embodiments, fibers may have at least one aspect ratio of at least 5:1, at least 10:1, at least 15:1, or even greater.

Elongated bone fibers may also be produced using the bone processing mill described in commonly assigned U.S. Pat. No. 5,607,269, the entire contents of which are incorporated herein by reference. Use of this bone mill results in the production of long, thin strips which quickly curl lengthwise to provide tube-like bone fibers. Elongated bone particles may be graded into different sizes to reduce or eliminate any less desirable size(s) of particles that may be present. In overall appearance, particles produced using this mill may be described as filaments, fibers, threads, slender or narrow strips, etc. In alternative embodiments, bone fibers and more evenly dimensioned particles may be produced by chipping, rolling, fracturing with liquid nitrogen, chiseling or planeing, broaching, cutting, or splitting along the axis (e.g., as wood is split with a wedge).

Alternatively or in addition, an entire bone section or relatively large portion of bone may be cut longitudinally into elongated sections using a band saw or a diamond-bladed saw. For example, the bone can be cut by making transverse cuts to prepare a bone section of the appropriate length, followed by longitudinal cuts using a band saw or a diamond cut saw. Elongated particles of bone can be further cut or machined into a variety of different shapes.

The bone particles employed in the composition can be powdered bone particles possessing a wide range of particle sizes ranging from relatively fine powders to coarse grains and even larger chips. Bone particles for use in the composites of the invention may have a length greater than 0.5 mm, for example, greater than 1 mm, greater than 2 mm, greater than 10 mm, greater than 100 mm, or greater than 200 mm, a thickness between 0.05 and 2 mm, for example, between 0.2 and 1 mm, and a width between 1 and 20 mm, for example, between 2 and 5 mm. Bone particles may be evenly dimensioned (e.g., having aspect ratios between 1:1 and 2:1) or may be elongated. In some embodiments, bone-derived particles may possess a median length to median thickness ratio of at least 2:1, at least 5:1, at least 10:1, at least 15:1, even greater, for example, at least 20:1, 30:1, 40:1, 50:1, or 100:1. In some embodiments, the ratio of length to thickness may range up to 500:1 or more. In addition, bone particles may have a median length to median width ratio of at least 2:1, at least 5:1, at least 10:1, at least 15:1, or even greater, for example, at least 20:1, 30:1, 40:1, 50:1, 100:1, or 200:1.

The bone particles may be sieved into different diameter sizes to eliminate any less desirable size(s) of fibers or more evenly dimensioned particles that may be present. In one embodiment, fibers collected from a milling machine may be lyophilized and manually sieved into a range of 300 μm to 500 μm in a particular cross-sectional dimension. One skilled in the art will recognize that the sieving method will determine what aspect must fall within 300-500 μm. Fiber length is independent of cross-sectional dimension and may be modified by adjusting the bit engagement length, the length of the bit in contact with the bone during the milling operation. Fibers may be an inch long or greater and may be as short as desired, depending on the desired aspect ratio. Fibers less than 50 μm long may increase the likelihood of inflammation depending on the tissues and how the implant degrades. Indeed, it may be desirable to include some volume or weight fraction of these fibers in a composite to stimulate a mild inflammatory response. Larger fibers may be further broken into smaller fibers by manually rolling them between the thumb and fingers and then sieved again to select the proper size fibers. Alternatively, fibers may be broken into smaller fibers by pressing or rolling.

The resulting fibers may have an aspect ratio of between 5:1 to 10:1. Broader or narrower fibers may be obtained by changing sieve grate sizes. Fibers with different widths and/or aspect ratios, may be obtained by adjusting the milling parameters, including sweep speed, bit engagement, rpm, cut depth, etc. In overall appearance, elongate bone particles can be described as filaments, fibers, threads, slender or narrow strips, etc. In some embodiments, at least about 60 weight percent, for example, at least about 75 weight percent or at least about 90 weight percent of the bone particles utilized in the preparation of the bone particle-containing composition herein are elongate

The bone particles may optionally be partially or completely demineralized in order to reduce their inorganic mineral content. Demineralization methods remove the inorganic mineral component of bone by employing acid solutions. Such methods are well known in the art, see for example, Reddi et al., Proc. Nat. Acad. Sci. 69, pp 1601-1605 (1972), incorporated herein by reference. The strength of the acid solution, the shape of the bone particles and the duration of the demineralization treatment will determine the extent of demineralization. Reference in this regard may be made to Lewandrowski et al., J Biomed Materials Res, 31, pp 365-372 (1996), also incorporated herein by reference.

In an exemplary demineralization procedure, the bone particles are subjected to a defatting/disinfecting step, which is followed by an acid demineralization step. An exemplary defatting/disinfectant solution is an aqueous solution of ethanol. Ordinarily, at least about 10 to about 40 percent by weight of water (i.e., about 60 to about 90 weight percent of defatting agent such as alcohol) should be present in the defatting/disinfecting solution to produce optimal lipid removal and disinfection within the shortest period of time. An exemplary concentration range of the defatting solution is from about 60 to about 85 weight percent alcohol and most preferably about 70 weight percent alcohol. Following defatting, the bone particles are immersed in acid over time to effect their demineralization. The acid also disinfects the bone by killing viruses, vegetative microorganisms, and spores. Acids that can be employed in this step include inorganic acids such as hydrochloric acid and organic acids such as peracetic acid. Alternative acids are well known to those skilled in the art. After acid treatment, the demineralized bone particles are rinsed with sterile water to remove residual amounts of acid and thereby raise the pH. The bone particles may be stored under aseptic conditions until they are used or sterilized using known methods shortly before incorporation into the composite. Additional demineralization methods are well known to those skilled in the art, for example, the method cited in Urist M R, A morphogenetic matrix for differentiation of bone tissue, Calcif Tissue Res. 1970; Suppl:98-101 and Urist M R, Bone: formation by autoinduction, Science. 1965 Nov. 12;150(698):893-9, the contents of both of which are incorporated herein by reference. Where elongate bone particles are employed, some entanglement of the wet demineralized bone particles will result. The wet demineralized bone particles can then be immediately shaped into any desired configuration or stored under aseptic conditions, advantageously in a lyophilized state, for processing at a later time. As an alternative to aseptic processing and storage, the particles can be shaped into a desired configuration and sterilized using known methods.

In an alternative embodiment, surfaces of bone particles may be lightly demineralized according to the procedures in our commonly owned U.S. patent application Ser. No. 10/285,715, published as U.S. Patent Publication No. 20030144743. Even minimal demineralization, for example, of less than 5% removal of the inorganic phase, exposes reactive surface groups such as hydroxyl and amine. Demineralization may be so minimal, for example, less than 1%, that the removal of the calcium phosphate phase is almost undetectable. Rather, the enhanced surface concentration of reactive groups defines the extent of demineralization. This may be measured, for example, by titrating the reactive groups. In one embodiment, in a polymerization reaction that utilizes the exposed allograft surfaces to initiate a reaction, the amount of unreacted monomer remaining may be used to estimate reactivity of the surfaces. Surface reactivity may be assessed by a surrogate mechanical test, such as a peel test of a treated coupon of bone adhering to a polymer. Alternatively or in addition, a portion of the surface of the bone particles may be so demineralized.

Mixtures or combinations of nondemineralized, superficially demineralized, partially demineralized, or fully demineralized bone particles can be employed. For example, one or more of the foregoing types of demineralized bone particles can be employed in combination with nondemineralized bone particles, i.e., bone particles that have not been subjected to a demineralization process.

Nondemineralized bone particles possess an initial and ongoing mechanical role, and later a biological role, in the osteoimplant. Nondemineralized bone particles act as a stiffener, providing strength to the osteoimplant and enhancing its ability to support load. These bone particles also play a biological role in bringing about new bone ingrowth by the process known as osteoconduction. Thus, these bone particles are gradually remodeled and replaced by new host bone as incorporation of the osteoimplant progresses over time.

The amount of each individual type of bone particle employed can vary widely depending on the mechanical and biological properties desired. Thus, mixtures of bone particles of various shapes, sizes, and/or degrees of demineralization may be assembled based on the desired mechanical, thermal, and biological properties of the composite. In addition or alternatively, composites may be formed having a single type of one particle or with multiple sections, each having a different type or mixture of bone particles. Suitable amounts of particle types can be readily determined by those skilled in the art on a case-by-case basis by routine experimentation.

If desired, the bone particles can be modified in one or more ways, e.g., their protein content can be augmented or modified as described in U.S. Pat. Nos. 4,743,259 and 4,902,296. Alternatively, the surface of a bone or ceramic particle may be treated to modify its surface composition. For example, nondemineralized bone particles may be rinsed with dilute phosphoric acid (e.g., for 1 to 15 minutes in a 5-50% solution by volume). Phosphoric acid reacts with the mineral component of the bone and coats the particles with dicalcium phosphate dihydrate. Treated surfaces may further be reacted with silane coupling agents as described in our copending application Ser. No. 10/681,651, the contents of which are incorporated herein by reference. Alternatively or in addition, bone or ceramic particles may be dried. For example, particles may be lyophilized for varying lengths of time, e.g., about 8 hours, about 12 hours, about 16 hours, about 20 hours, or a day or longer. Moisture may be removed by heating the particles to an elevated temperature, for example, 60° C., 70° C., 80° C., or 90° C., with or without a dessicant. In another embodiment, deorganified bone particles may be used. Deorganified bone particles may be obtained commercially, for example, BIO-OSS™ from Osteohealth, Co. or OSTEOGRAF™ from Dentsply. Alternatively or in addition, bone particles may be partially or completely deorganified using techniques known to those skilled in the art, such as incubation in 5.25% sodium hypochlorite.

Crosslinking can be performed in order to improve the strength of the osteoimplant. Crosslinking of the bone particle-containing composition can be effected by a variety of known methods including chemical reaction, the application of energy such as radiant energy, which includes irradiation by UV light or microwave energy, drying and/or heating and dye-mediated photo-oxidation; dehydrothermal treatment in which water is slowly removed while the bone particles are subjected to a vacuum; and, enzymatic treatment to form chemical linkages at any collagen-collagen interface. The preferred method of forming chemical linkages is by chemical reaction.

Chemical crosslinking agents include those that contain bifunctional or multifunctional reactive groups, and which react with surface-exposed collagen of adjacent bone particles within the bone particle-containing composition. By reacting with multiple functional groups on the same or different collagen molecules, the chemical crosslinking agent increases the mechanical strength of the osteoimplant.

Chemical crosslinking involves exposing the bone particles presenting surface-exposed collagen to the chemical crosslinking agent, either by contacting bone particles with a solution of the chemical crosslinking agent, or by exposing bone particles to the vapors of the chemical crosslinking agent under conditions appropriate for the particular type of crosslinking reaction. For example, the osteoimplant can be immersed in a solution of cross-linking agent for a period of time sufficient to allow complete penetration of the solution into the osteoimplant. Crosslinking conditions include an appropriate pH and temperature, and times ranging from minutes to days, depending upon the level of crosslinking desired, and the activity of the chemical crosslinking agent. The resulting osteoimplant is then washed to remove all leachable traces of the chemical.

Suitable chemical crosslinking agents include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as other heterobifunctional crosslinking agents; hexarnethylene diisocyante; sugars, including glucose, will also crosslink collagen.

Glutaraldehyde crosslinked biomaterials have a tendency to over-calcify in the body. In this situation, should it be deemed necessary, calcification-controlling agents can be used with aldehyde crosslinking agents. These calcification-controlling agents include dimethyl sulfoxide (DMSO), surfactants, diphosphonates, aminooleic acid, and metallic ions, for example ions of iron and aluminum. The concentrations of these calcification-controlling agents can be determined by routine experimentation by those skilled in the art.

When enzymatic treatment is employed, useful enzymes include those known in the art which are capable of catalyzing crosslinking reactions on proteins or peptides, preferably collagen molecules, e.g., transglutaminase as described in Jurgensen et al., The Journal of Bone and Joint Surgery, 79-a (2), 185-193 (1997), herein incorporated by reference.

Formation of chemical linkages can also be accomplished by the application of energy. One way to form chemical linkages by application of energy is to use methods known to form highly reactive oxygen ions generated from atmospheric gas, which in turn, promote oxygen crosslinks between surface-exposed collagen. Such methods include using energy in the form of ultraviolet light, microwave energy and the like. Another method utilizing the application of energy is a process known as dye-mediated photo-oxidation in which a chemical dye under the action of visible light is used to crosslink surface-exposed collagen.

Another method for the formation of chemical linkages is by dehydrothermal treatment, which uses combined heat and the slow removal of water, preferably under vacuum, to achieve crosslinking of bone particles. The process involves chemically combining a hydroxy group from a functional group of one collagen molecule and a hydrogen ion from a functional group of another collagen molecule reacting to form water which is then removed resulting in the formation of a bond between the collagen molecules.

Inorganic ceramic materials may also be employed, either alone or in combination with bone, to form composites. For example, non-bony calcium phosphate materials may also be exploited for use with the invention. Exemplary inorganic ceramics for use with the invention include calcium carbonate, calcium sulfate, calcium phosphosilicate, sodium phosphate, calcium aluminate, calcium phosphate, hydroxyapatite, α-tricalcium phosphate, dicalcium phosphate, β-tricalcium phosphate, tetracalcium phosphate, amorphous calcium phosphate, octacalcium phosphate, and BIOGLASS™, a calcium phosphate silica glass available from U.S. Biomaterials Corporation. Substituted CaP phases are also contemplated for use with the invention, including but not limited to fluorapatite, chlorapatite, Mg-substituted tricalcium phosphate, and carbonate hydroxyapatite. Additional calcium phosphate phases suitable for use with the invention include those disclosed in U.S. Patents Nos. RE 33,161 and RE 33,221 to Brown et al.; U.S. Pat. Nos. 4,880,610; 5,034,059; 5,047,031; 5,053,212; 5,129,905; 5,336,264; and 6,002,065 to Constantz et al.; U.S. Pat. Nos. 5,149,368; 5,262,166 and 5,462,722 to Liu et al.; U.S. Pat. Nos. 5,525,148and 5,542,973 to Chow et al., U.S. Pat. Nos. 5,717,006 and 6,001,394 to Daculsi et al., U.S. Pat. No. 5,605,713 to Boltong et al., U.S. Pat. No. 5,650,176 to Lee et al., and U.S. Pat. No. 6,206,957 to Driessens et al, and biologically-derived or biomimetic materials such as those identified in Lowenstam H A, Weiner S, On Biomineralization, Oxford University Press, 234 pp. 1989, incorporated herein by reference. Non-calcium ceramics such as alumina or zirconia are also appropriate for use according to the teachings herein.

Alternatively or in addition, metallic materials may also be employed in composites or in the implant components. Exemplary materials include titanium and titanium alloy fibers such as NiTi (shape memory materials) and Ti-6Al-4V. Additional metallic materials include biocompatible steels and cobalt-chromium-molybdenum alloys. Radio-opaque materials may be included in composites or in the stacking units to facilitate long term evaluation of a patient's progress.

The dimensions of the various natural, recombinant, and synthetic materials making up a composite may vary widely depending on the dimensions of the implant site. In one embodiment, these dimensions may range from about 1 cm to about 1 meter in length, for example, from about 3 cm to about 8 cm in length, from about 0.5 mm to about 30 mm in thickness, for example, from about 2 mm to about 10 mm in thickness, and from about 0.05 mm to about 150 mm in width, for example, from about 2 mm to about 10 mm in width.

Any biocompatible polymer may be used to form composites for use according to the invention. As noted above, the cross-link density and molecular weight of the polymer may need to be manipulated so that the polymer may be formed and set when desired. A number of biodegradable/resorbable and non-biodegradable/non-resorbable biocompatible polymers are known in the field of polymeric biomaterials, controlled drug release and tissue engineering (see, for example, U.S. Pat. Nos. 6,123,727, 5,804,178, 5,770,417, 5,736,372, and 5,716,404 to Vacanti; U.S. Pat. Nos. 6,095,148 and 5,837,752 to Shastri; U.S. Pat. No. 5,902,599 to Anseth; U.S. Pat. Nos. 5,696,175, 5,514,378, and 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621 and 4,638,045 to Kohn; see also Langer, Acc. Chem. Res. 33:94, 2000; Langer, J. Control Release 62:7, 1999; and Uhrich et al., Chem. Rev. 99:3181, 1999).

A wide variety of biocompatible polymers are known in the art. In one embodiment, the polymer is also biodegradable/resorbable. Suitable biodegradable/resorbable polymers are well known in the art and include collagen-GAG, collagen, oxidized cellulose, alginic acid, cotton, catgut, silk, fibrin, elastin, starches, lactide-glycolide copolymers in any ratio, e.g., 85:15, 40:60, 30:70, 25:75, or 20:80, poly(L-lactide-co-D,L-lactide), polylactide, polyglycolide, poly(lactide-co-glycolide), polydioxanone, poly(epsilon caprolactone-co-p-dioxanone), polycarbonates, polyhydroxybutyrate, polyhydroxyvalyrate, poly(propylene glycol-co-fumaric acid), polyhydroxyalkanoates, polyphosphazenes, poly(alkylcyanoacrylates), degradable hydrogels, polyoxamers, polyarylates, amino-acid derived polymers, amino-acid-based polymers, amino-acid-based polymers, particularly tyrosine-based polymers, including tyrosine-based polycarbonates and polyarylates, pharmaceutical tablet binders (such as Eudragit® binders available from Hüls America, Inc.), polyvinylpyrrolidone, cellulose, ethyl cellulose, micro-crystalline cellulose and blends thereof; starch ethylenevinyl alcohols, poly(anhydrides), poly(hydroxy acids), poly(ortho esters), poly(propylfumerates), poly(caprolactones), polyamides, polyamino acids, polyacetals biodegradable polycyanoacrylates, biodegradable polyurethanes and natural and modified polysaccharides. Exemplary tyrosine-based polymers include, but are not limited to, tyrosine based polycarbonates and polyarylates such as those described by U.S. Pat. Nos. 5,587,507, 5,670,602, and 6,120,491, such as poly(desaminotyrosyltyrosine(ethyl ester) carbonate) (PolyDTE carbonate), poly(desaminotyrosyltyrosine carbonate) (PolyDT carbonate), and co-polymers of these in ratios of, e.g., 25:75, 40:60, 60:40, or 75:25. Additional polymers include bioabsorbable block copolymers made of hard phase forming monomers, e.g., glycolide and lactide, and soft phase monomers, e.g., 1,4 dioxane-2-one and caprolactone, as described, e.g., in U.S. Pat. No. 5,522,841, incorporated herein by reference.

Synthetic and recombinant versions or modified versions of natural polymers may also be used. Exemplary synthetic ECM analogs include silk-elastin polymers produced by Protein Polymer Technologies (San Diego, Calif.) and BioSteel™, a recombinant spider silk produced by Nexia Biotechnologies (Vaudrevil-Dorion, QC, Canada). Recombinant fibers may be obtained from microorganisms, for example, genetically engineered microorganisms such as yeast and bacteria and genetically engineered eucaryotic cell cultures such as Chinese hamster ovary cell lines, HeLa cells, etc. For example, U.S. Pat. Nos. 5,243,038 and 5,989,894, each of which is incorporated herein by reference, describe the expression of spider silk protein, collagen proteins, keratins, etc., using genetically engineered microorganisms and eucaryotic cell lines.

Non-biodegradable/non-resorbable polymers may also be used as well. For example, polypyrrole, polyanilines, polythiophene, and derivatives thereof are useful electroactive polymers that can transmit voltage from the endogenous bone to the implant. Bone is piezoelectric, and voltage within the bone may help it maintain the proper shape as it remodels. Other non-biodegradable, yet biocompatible polymers include polystyrene, polyesters, polyureas, poly(vinyl alcohol), polyamides, poly(tetrafluoroethylene), and expanded polytetrafluroethylene (ePTFE), poly(ethylene vinyl acetate), polypropylene, polyacrylate, non-biodegradable polycyanoacrylates, non-biodegradable polyurethanes, mixtures and copolymers of poly(ethyl methacrylate) with tetrahydrofurfuryl methacrylate, polymethacrylate, poly(methyl methacrylate), polyethylene, including ultra high molecular weight polyethylene (UHMWPE), polypyrrole, polyanilines, polythiophene, poly(ethylene oxide), poly(ethylene oxide co-butylene terephthalate), poly ether-ether ketones (PEEK), and polyetherketoneketones (PEKK).

Additional polymeric binders include those described in U.S. Pat. Nos. 5,216,115; 5,317,077; 5,587,507; 5,658,995; 5,670,602; 5,695,761; 5,981,541; 6,048,521; 6,103,255; 6,120,491; 6,284,862; 6,319,492; and, 6,337,198, all of which are incorporated herein by reference. The polymeric binders described in these patents include amino acid-derived polycarbonates, amino acid-derived polyarylates, polyarylates derived from certain dicarboxylic acids and amino acid-derived diphenols, anionic polymers derived from L-tyrosine, polyarylate random block copolymers, polycarbonates, poly(α-hydroycarboxylic acids), poly(caprolactones), poly(hydroxybutyrates), polyanhydrides, poly(ortho esters), polyesters and bisphenol-A based poly(phosphoesters). Additional suitable polymeric binders are the copolymers of polyalkylene glycol and polyester of U.S. Patent Application Publication 2001/0051832, the polyester resin formed in situ from a liquid mixture of crosslinkable polyester and crosslinking agent as described in U.S. Pat. No. 4,722,948, and the polymerizable polymeric binder-forming materials described in U.S. Pat. No. 6,352,667, all three of which references are incorporated herein by reference. Those skilled in the art will recognize that this is an exemplary, not a comprehensive, list of polymers appropriate for in vivo applications.

These polymers and the monomers that are used to produce any of these polymers are easily purchased from companies such as Polysciences (Warrington, Pa.), Sigma-Aldrich (St. Louis, Mo.), and Scientific Polymer Products (Ontario, N.Y.). Those skilled in the art will recognize that this is an exemplary, not a comprehensive, list of polymers appropriate for in vivo applications. Co-polymers and/or blends of the above polymers may also be used with the invention.

Natural and recombinant fibers may be modified in a variety of ways before being incorporated into an aggregate. For example, fibrous tissues may be frayed to expose protein chains and increase the surface area of the tissue. Rinsing fibrous tissue or partially demineralized bone particles in an alkaline solution, or simply partially demineralizing bone particles, will fray fibrous proteins within the tissue. For example, bone fibers may be suspended in aqueous solution at a pH of about 10 for about 8 hours, after which the solution is neutralized. One skilled in the art will recognize that this time period may be increased or decreased to adjust the extent of fraying. Agitation, for example, in an ultrasonic bath, may assist in fraying and/or separating collagen fibers, as well as improving penetration of acidic, basic, or other fluids, especially for bony tissues. Alternatively or in addition, bone or inorganic calcium phosphate particles may be mechanically stirred, tumbled, or shaken, with or without the addition of abrasives.

Polymers and fibrous tissues, especially those containing collagen, such as bone and tendon, may be cross-linked before incorporation into a composite. A variety of cross-linking techniques suitable for medical applications are well known in the art. For example, compounds like 1-ethyl-3-(3-dimethylaminopropyl) carbodiimide hydrochloride, either alone or in combination with N-hydroxysuccinimide (NHS) will crosslink collagen at physiologic or slightly acidic pH (e.g., in pH 5.4 MES buffer). Acyl azides and genipin, a naturally occurring bicyclic compound including both carboxylate and hydroxyl groups, may also be used to cross-link collagen chains (see Simmons, et al, “Evaluation of collagen cross-linking techniques for the stabilization of tissue matrices,” Biotechnol. Appl. Biochem., 1993, 17:23-29; PCT Publication WO98/19718, the contents of both of which are incorporated herein by reference). Alternatively, hydroxymethyl phosphine groups on collagen may be reacted with the primary and secondary amines on neighboring chains (see U.S. Pat. No. 5,948,386, the entire contents of which are incorporated herein by reference). Standard cross-linking agents such as mono- and dialdehydes, polyepoxy compounds, tanning agents including polyvalent metallic oxides, organic tannins, and other plant derived phenolic oxides, chemicals for esterification or carboxyl groups followed by reaction with hydrazide to form activated acyl azide groups, dicyclohexyl carbodiimide and its derivatives and other heterobifunctional crosslinking agents, hexamethylene diisocyanate, ionizing radiation, and sugars may also be used to cross-link fibrous tissues and polymers. The tissue is then washed to remove all leachable traces of the material. Enzymatic cross-linking agents may also be used. One skilled in the art will easily be able to determine the optimal concentrations of cross-linking agents and incubation times for the desired degree of cross-linking.

The composite may include practically any ratio of polymer and bone, for example, between about 5 weight % polymer and about 90 weight % polymer. For example, the composite may include about 25% to about 30% polymer or approximately equal weights of polymer and bone. The proportions of the polymer and bone will influence both the mechanical properties of the composite, including fatigue, strain, compressive strength, and the degradation rate of the composite. In addition, the cellular response to the composite will vary with the proportion of polymer and bone. One skilled in the art will recognize that standard experimental techniques may be used to test these properties for a range of compositions to optimize a composite for a desired application. For example, standard mechanical testing instruments may be used to test the compressive strength and stiffness of the composite. Cells may be cultured on the composite for an appropriate period of time and the metabolic products and the amount of proliferation (e.g., the number of cells in comparison to the number of cells seeded) analyzed. The weight change of the composite may be measured after incubation in saline or other fluids. Repeated analysis will demonstrate whether degradation is linear or not, and mechanical testing of the incubated material will show the change in mechanical properties as the composite degrades.

Biologically active materials, including biomolecules, small molecules, and bioactive agents may also be combined with the polymer and bone to, for example, stimulate particular metabolic functions, recruit cells, or reduce inflammation. For example, DNA vectors that will be taken up by the patient's cells and cause the production of growth factors such as bone morphogenetic protein may also be included in the composite. These materials need not be covalently bonded to any component of the composite. A material may be selectively distributed on or near the surface of the composite using the layering techniques described above. While the surface of the composite will be mixed somewhat as the composite is manipulated in the implant site, the thickness of the layer will ensure that at least a portion of the surface layer of the composite remains at the surface of the stacking unit. Alternatively or in addition, biologically active components may be covalently linked to the bone or polymer particles before combination. For example, silane coupling agents having amine, carboxyl, hydroxyl, or mercapto groups may be attached to the bone particles through the silane and then to reactive groups on a biomolecule, small molecule, or bioactive agent. Composites may contain radiopaque, radiographic additives or vary in density from normal bone such that they are easily visualized upon radiography.

The composite may be formed, machined, or both, into a variety of shapes. In addition to the shapes described above, exemplary shapes that can be created include, without limitation, a sheet, plate, particle, sphere, strand, coiled strand, coiled coil, capillary network, film, fiber, mesh, disk, cone, rod, cup, pin, screw, tube, tooth, tooth root, bone or portion of bone, wedge or portion of wedge, cylinder, and threaded cylinder. In one embodiment, the composite is formed in a mold having the shape of a desired stacking unit, such as the flat plates, caps, and wedges described above. The forming of various stacking unit shapes may be accomplished by injection, pressing, and/or packing the composite into molds or forms. The stacking units are then solidified by any practical means (e.g., by thermosetting, polymerization or crosslinking). Alternatively, the composite may be molded into a block (e.g., a cylinder) that is machined into a desired shape. The composite may be machined in either its set condition or its formable condition.

Alternative techniques are also available for producing stacking units. In one embodiment, bone-derived particles are combined with a solvent to form a precursor. Since the solvent will usually be removed, it does not have to be non-toxic; however, a biocompatible solvent is preferred. Exemplary solvents include water, lower alkanols, ketones, and ethers and mixtures of any of these. The precursor may then extruded at an appropriate temperature and pressure to produce a disc or other implant component. The precursor may be shaped by thermal or chemical bonding, or both. In one embodiment, a portion of the solvent is removed from the precursor before extrusion. Alternatively or in addition, the precursor material may be molded. A variety of materials processing methods will be well known to those skilled in the art. For example, the precursor material may be molded using a press such as a Carver press to create a component having a particular shape.

In an alternative embodiment employing a precursor of bone particles and a solvent, a binding agent is included in the precursor either before or after forming the aggregate. For example, the bone particles and binding agent solution may be combined in a slurry or formed into a green body. The precursor, including the binding agent, may be cast, molded, extruded, or otherwise processed as discussed above.

The binding agent links adjacent bone particles either directly or by forming bridge-like structures between them. In one embodiment, inorganic binding agents include a metal oxide, metal hydroxide, metal salt of an inorganic or organic acid, or a metal containing silica-based glass. The metal may be endogenous (e.g., bone derived calcium) or exogenous. The metal may be divalent, for example, an alkaline earth metal, e.g., calcium. A variety of appropriate solvents and binding agents are disclosed in our commonly owned U.S. Pat. No. 6,478,825, the entire contents of which are incorporated herein by reference. In one embodiment, the binding agent is at least slightly soluble in a polar solvent to promote precipitation. Since the solvent will usually be removed to precipitate the binding agent on the surfaces of the bone derived elements, the solvent does not have to be non-toxic; however, a biocompatible solvent is preferred. Exemplary solvents include water, lower alkanols, ketones, and ethers and mixtures of any of these.

Where elongated particles are used in an extruded composite, they will tend to be aligned roughly parallel to one another. This may be exploited by extruding composites to form stacking units in different orientations. That is, stacking units of roughly the same shape may be produced with different orientations of the elongated particles, so the direction of the particles within the assembled group of stacking units varies across the implant (as in plywood), improving the ability of the assembled group of stacking units to withstand different loading modes.

The composite material may be formed by a variety of techniques in addition to those described above. For example, bone or ceramic particles may be combined with a relatively flowable polymer that is then set to form a solid composite, as described in our co-pending application number Ser. No. 10/735,135, entitled “Formable and settable polymer bone composite and method of production thereof,” published as U.S. Patent Publication No. 20050008672, the entire contents of which are incorporated herein by reference. In an alternative embodiment, bone or ceramic particles are combined with a monomer or a polymer precursor that is polymerized to create a composite material, as disclosed in our co-pending applications Ser. No. 10/639,912, entitled “Synthesis of a bone-polymer composite material,” published as U.S. Patent Publication No. 20040146543, and 10/771,736, entitled “Polyurethanes for Osteoimplants,” the entire contents of both of which are incorporated herein by reference. The modifications to the ceramic and bone particles described above enhance their reactivity and facilitate the formation of chemical bonds between the particles and the polymer, increasing the interfacial strength of the composite and increasing the extent to which the included particles can contribute to the overall mechanical properties of the composite. Alternatively, or in addition, a coupling agent may be added to the bone or ceramic particles to add chemical functionality that can co-polymerize with the monomer, as disclosed in our co-pending application Ser. No. 10/681,651, entitled “Coupling agents for orthopedic biomaterials,” published as U.S. Patent Publication No. 20050008620, the entire contents of which are incorporated herein by reference. The composite fabrication techniques and compositions disclosed in commonly owned U.S. Pat. Nos. 5,899,939, 6,123,731, 6,294,187, and 6,440,044, the contents of all of which are incorporated herein by reference, are also appropriate for the production of stacking units.

In another embodiment, stacking units may be fabricated in a manner that is intended to facilitate bony ingrowth and cellular infiltration into the composite while maintaining the mechanical strength of the material within the implant site. By carefully evaluating the volume fraction of cell conducting material for use in a composite, stacking units may be fabricated that provide paths for tissue penetration into the component, even where there is no porosity to promote cell migration. Techniques for determining the appropriate proportion of cell conducting materials and fabricating suitable composites are described in our pending application, filed on the same day as the current application using Express Mail No. EL979824567US, the entire contents of which are incorporated herein by reference.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A system for inducing fusion of vertebrae, comprising:
a plurality of stacking inserts for placement in an intervertebral space, each insert comprising a composite consisting essentially of bone fragments embedded in a biocompatible polymer, the composite having osteogenic properties,
wherein a subset of said plurality of stacking inserts may be selected to fit the dimensions of the intervertebral space.

2. The system of claim 1, wherein the biocompatible polymer is biodegradable.

3. The system of claim 1, wherein the biocompatible polymer is selected from the group consisting of collagen-GAG, collagen, oxidized cellulose, fibrin, elastin, starches, polylactic acid, polyglycolic acid, polylactic-co-glycolic acid, polylactide, polyglycolide, poly(lactide-co-glycolide), polydioxanone, polycarbonates, polyhydroxybutyrate, polyhydroxyvalyrate, poly(propylene glycol-co-fumaric acid), polyhydroxyalkanoates, polyphosphazenes, poly(alkylcyanoacrylates), degradable hydrogels, poloxamers, polyarylates, amino-acid derived polymers, amino-acid-based polymers, amino-acid-based polymers, tyrosine-based polymers, tyrosine-based polycarbonates and polyarylates, pharmaceutical tablet binders, polyvinylpyrrolidone, cellulose, ethyl cellulose, micro-crystalline cellulose and blends thereof, starch ethylenevinyl alcohols, poly(anhydrides), poly(hydroxy acids), poly(ortho esters), poly(propylfumerates), poly(caprolactones), polyamides, polyamino acids, polyacetals biodegradable polycyanoacrylates, biodegradable polyurethanes, natural and modified polysaccharides, recombinant versions of biological polymers, silk-elastin, polypyrrole, polyanilines, polythiophene, polystyrene, polyesters, non-biodegradable polyurethanes, polyureas, polyamides, poly(tetrafluoroethylene), poly(ethylene vinyl acetate), polypropylene, polyacrylate, polymethacrylate, poly(methyl methacrylate), polyethylene, poly(ethylene oxide), amino acid-derived polycarbonates, amino acid-derived polyarylates, polyarylates derived from certain dicarboxylic acids and amino acid-derived diphenols, anionic polymers derived from L-tyrosine, polyarylate random block copolymers, polycarbonates, poly(α-hydroycarboxylic acids), poly(caprolactones), poly(hydroxybutyrates), polyanhydrides, poly(ortho esters), polyesters, bisphenol-A based poly(phosphoesters), copolymers of polyalkylene glycol and polyester, and derivatives and combinations of any of the above.

4. The system of claim 1, wherein the biocompatible polymer is electroactive.

5. The system of claim 1, wherein the inserts are stacked vertically, laterally horizontally, horizontally along the anterior-posterior axis, or diagonally with respect to the intervertebral space.

6. The system of claim 1, wherein the bone particles are nondemineralized.

7. The system of claim 1, wherein the bone particles are partially or fully demineralized.

8. The system of claim 1, wherein the bone particles are obtained from a member of the group consisting of cortical bone, cancellous bone, cortico-cancellous bone, and mixtures thereof.

9. The system of claim 1, wherein the bone particles are obtained from a member of the group consisting of autogenous bone, allogenic bone, xenogenic bone, and mixtures thereof.

10. The system of claim 1, wherein the bone particles represent about 50%-90% by weight of the composite.

11. The system of claim 1, wherein the bone particles represent about 60%-80% by weight of the composite.

12. The system of claim 1, wherein the bone particles represent about 70%-75% by weight of the composite.

13. The system of claim 1, wherein at least a portion of the inserts have parallel top and bottom surfaces.

14. The system of claim 1, wherein at least a portion of the inserts have a wedge-shaped cross-section.

15. The system of claim 1, wherein at least a portion of the inserts are in the form of a partial or complete spherical cap.

16. The system of claim 1, wherein the inserts comprise connecting structures, surface texture, or both, that inhibit relative movement between the inserts when deployed in the intervertebral space.

17. The system of claim 16, wherein the connecting structures are selected from the group consisting of ridges, teeth, threads, wedges, bumps, cylinders, pyramids, blocks, valleys, dimples, holes, grids, mortises, tenons, tongues, grooves, valleys, troughs, dimples, pits, and dovetails.

18. The system of claim 16, wherein the securing structures can be used to attach adjacent inserts, and wherein the structures provide audible or tactile feedback when attachment occurs.

19. The system of claim 1, wherein the inserts comprise securing structures that inhibit movement of the inserts relative to vertebrae adjacent to the inserts.

20. The system of claim 19, wherein the securing structures are selected from the group consisting of ridges, bumps, cylinders, pyramids, blocks, valleys, dimples, holes, and grids.

21. The system of claim 1, further comprising a fastener for connecting inserts to one another.

22. The system of claim 21, wherein the fastener is selected from the group consisting of screws, rivets, biscuits, rabbets, dowels, and extensible structures that lock around a set of inserts.

23. The system of claim 21, wherein at least a portion of the inserts comprise predrilled holes, slots, or notches sized to accommodate the fastener.

24. The system of claim 1, further comprising a pedicle screw that prevents relative motion of vertebrae forming the intervertebral space.

25. A method of fusing adjacent vertebrae, comprising:
inserting into an intervertebral space defined by the adjacent vertebrae a plurality of inserts that together match the size and shape of the intervertebral cavity, wherein the inserts comprise a composite consisting essentially of bone fragments embedded in a biocompatible polymer, the composite having osteogenic properties.

26-48. (canceled)
